(19) United States
(12) Patent Application Publication
(10) Pub. No.: US 2008/0188001 A1
(43) Pub. Date: Aug. 7, 2008

(54) Methods For Gene Targeting

(76) Inventor: Michael Paul Strathmann, Seattle, WA (US)

Correspondence Address: MICHAEL STRATHMANN, 1205 8TH AVE W, SEATTLE, WA, 98119, US

(21) Appl. No.: 12/027,199

(22) Filed: Feb. 6, 2008

Related U.S. Application Data
(60) Provisional application No. 60/888,529, filed on Feb. 6, 2007.

(51) Int. Cl. C12N 15/87

(52) U.S. Cl. 435/463

(57) ABSTRACT

The present invention provides methods for generating and characterizing gene targeting events by using tags. More specifically, the invention employs methods to enrich for cells that have undergone the desired targeting event.

### 1. RELATED APPLICATION DATA

This application claims the benefit of U.S. Provisional Application No. 60/888,529 filed on Feb. 6, 2007, which is incorporated herein by reference.

### 2. FIELD OF THE INVENTION

The present invention is related to the field of molecular biology, and provides methods for disrupting and modifying genes.

### 3. BACKGROUND

Two methods are commonly used to disrupt or “knock out” a gene in a cell: homologous recombination and gene trapping. Homologous recombination is usually performed by creating a construct which is derived from the gene in vitro using standard recombinant techniques. The construct is introduced into the cell by transfection, transformation, etc. At some frequency, the cellular machinery recombines the introduced construct with homologous sequences in the chromosome thereby disrupting the gene. Various selection methods may be utilized to select or screen cells for the rare recombination event (Capecchi, M. R., Science, 244:1288-1292, 1989; Capecchi, M. R. et al., U.S. Pat. No. 5,464,764). Typically, one gene at a time may be disrupted by homologous recombination.

Gene trapping involves the nonspecific insertion of DNA (an insertion element), which carries a selectable marker, into a chromosome. If the DNA is inserted into a gene, the gene may be disrupted. Subsequent steps in the protocol entail analysis of the insertion site to determine if a gene of interest has been disrupted. Typically, many cells containing independent insertions will be analyzed to produce a large collection of gene knock-outs. The selectable marker is frequently introduced by an engineered retrovirus or transposon. Various selections may be employed to enrich for insertions into genes, e.g. promoter trapping, poly-A trapping, etc. (Zambrowicz, B. et al., U.S. Pat. No. 6,080,576; Tessier-Lavigne, M. et al., U.S. Pat. No. 6,248,934; Ishida Y. et al, Nucleic Acids Res., 27:e35, 1999; Durick, K. et al., Genome Res., 9:1019-1025, 2007).

Homologous recombination is a technique more suited to analyzing a small number of specific genes because of the upfront labor required to create gene specific constructs and the subsequent labor necessary to isolate the cells that have undergone the correct recombination event. Gene trapping is more suited to analyzing larger numbers of genes that are not determined at the outset. The randomness of the integration process limits the likelihood that any one gene will be disrupted unless a very large number of integration events are examined. The amount of effort required to disrupt all the genes in a cell or organism is so prohibitive at present that only a consortium of well funded scientists would undertake the task.

What is needed in the art is a general method for disrupting genes in a cell that is simple and inexpensive enough to target specific genes and reduce the cost and effort to disrupt all or a large number of genes in a cell or organism. The instant invention describes such a method.

### 4. BRIEF DESCRIPTION OF THE FIGURES

FIG. 1 is a drawing of a preferred embodiment of a gene trap vector and the integration of the vector into a gene.

FIG. 2 is a drawing of a preferred embodiment of a construct for targeting a gene by homologous recombination and the resulting recombination product.

### 5. SUMMARY

It is an object of the invention to provide methods for gene targeting. The invention provides methods for generating and characterizing gene targeting events by using tags. More specifically, the method employs RNAi and other tag-specific selections to enrich for cells that have undergone the desired targeting event.

### 6. DETAILED DESCRIPTION OF PREFERRED EMBODIMENTS

Strathmann previously described a method for generating a collection of cells wherein many of the cells contain tagged insertion elements (Strathmann, M., U.S. Pat. No. 6,480,791, which is hereby incorporated in its entirety). The tag is a stretch of sequence within the insertion element that is unique to that cell or more accurately that clonal population of cells (cell clone) within the collection. A collection of cell clones is generated for example by randomly inserting the tagged insertion elements into the genome so that usually any one cell (or organism) preferably will have undergone only one integration event. These cells can be spatially separated. For example, mammalian cells can be infected with a collection of tagged retroviral vectors. Each vector may contain a reporter gene (e.g., GFP). The transfected cells (that is, the cells that express the reporter gene) can be spatially separated from each other and from uninfected cells by flow cytometry and cell sorting (see Galbraith, D. W. et al., Methods Cell Biol., 58:315-41, 1999), or by other means. Though this example is directed towards random integration events, the method is equally applicable to “targeted” integration events. For example, insertion elements have been described that target the integration events to genes by providing selectable markers that lack promoters, or must be properly spliced to function, etc. (e.g. Sedivy, J. M. et al., Proc. Natl. Acad. Sci. USA, 86:227-31, 1989; Friedrich, G. et al., Genes Dev., 5:1513-23, 1991; Skarnes, W. C. et al., Proc. Natl. Acad. Sci. USA, 92:6592-6, 1995; Ruley, H. E. et al., U.S. Pat. No. 5,627,058, 1997; Sands, A. et al., PCT Pat. Pub. No. WO 98/14614, 1998).

The location of a tagged insertion element in the genome can be determined by rescuing one or both junctions (i.e. the genomic DNA that flanks the insertion element) along with the tag (the tagged junctions) by methods well known in the art (see for example Strathmann, M., U.S. Pat. No. 6,480,791, and references therein). By sequencing the rescued tag and junctions, it is possible to establish the identity of the tag and the location of the associated insertion element within the genome. cDNA may also be used to rescue some junctions, but if the insertion element resides within an intron, then the precise location may not be evident from cDNA sequence due to splicing. Enormous economies of scale may be achieved by rescuing all the tagged junctions from a pooled collection of cells containing tagged insertion elements at different positions. The sequence of all the tags and their associated junctions may be determined simultaneously using a massively parallel sequencing platform such as 454 Life Sciences, Solexa, etc., (Margulies, M. et al., Nature, 437:376-80, 2005). For example, one or a small number of sequencing primers that hybridize to a common region in the insertion element may be used with the 454 Life Sciences' machine to sequence through the tags into the genomic DNA at the junctions. Consider, for example, a typical retrovirus vector (e.g. RET, Ishida Y. et al, Nucleic Acids Res., 27:e35, 1999; Shigeoka T. et al., Nucleic Acids Res. 33:e20, 2005) that is used in gene trap experiments. A large collection of tagged vectors can be easily created using standard recombinant techniques by ligating a large collection of oligonucleotides of random sequence (e.g. 25-mers) into a restriction site within the vector so that any one vector incorporates one oligonucleotide. The sequence of this oligonucleotide is the tag sequence. This collection of tagged vectors may be transfected en masse into a packaging cell line to produce virus particles. The virus particles are combined with a cell line such that many cells are infected with one or no virus particles. Cells harboring an integrated provirus are selected using standard procedures. The cells may be clonally expanded, for example in individual wells of a microtiter plate, or the entire population may be maintained as a single culture. In this way, a collection of cells comprising tagged insertion elements is created. If the cells are maintained in individual wells, the cells or nucleic acid from the cells may be pooled prior to rescuing the junctions. Obviously, this step is not needed if the cells are maintained as a single culture. The rescued collection of tagged junctions may then be subjected to massively parallel sequencing to determine the identity of the tag and the location of the tagged insertion element. Some of the insertion elements will reside within genes in such a way that the function of the genes are disrupted.

Some methods for massively-parallel sequencing do not produce very long sequencing reads (e.g. the Genome Sequencer FLX from 454 Life Sciences and Roche Applied Science has an average read length between 200 and 300 bases, while Solexa's instrument can read 30-35 bases). If reads are short and a retroviral vector is utilized to introduce the tagged marker, it is possible to use a poly-A trap vector and sequence cDNA as long as the tag is positioned just upstream of the splice-donor site. In this way, the tag will be positioned very near the endogenous splice-acceptor and any intervening retroviral sequences will be removed as part of the intron. Alternatively, the tag may be positioned near a restriction site and junctions may be rescued from genomic DNA by circularizing the DNA, which joins this restriction site to genomic DNA at some distance from the retroviral LTR (long terminal repeat). This method is analogous to paired-end sequencing protocols that have been developed by the instrument makers (see for example Korbel, J. O., Science 318:420-426, 2007). The Genome Sequencer FLX may be capable of sequencing junctions rescued from genomic DNA with a first sequencing primer in the retroviral LTR and then sequencing through the tag with a second primer. The second sequence may be determined after denaturing and removing the first sequencing product or by simply terminating the first sequencing product with a dideoxy nucleotide prior to annealing the second primer to initiate the sequencing reaction from a second site.

While there is utility in having a collection of cells comprising tagged insertion elements in known locations (see for example, Mazurkiewicz P. et al., Nat. Rev. Genet. 7:929-39, 2006; Smith, V. et al, Proc. Natl. Acad. Sci USA, 92:6479-83, 1995; Ross-Macdonald, P. et al, Nature 402:362-3, 1999; Chun, K. T. et al., Yeast 13:233-40, 1997), a great deal more information can be learned by isolating a cell clone comprising one tagged insertion element (or a small number). If the population of cells described above was originally stored as cell clones in separate wells of a microtiter plate then each tag can be associated with a cell clone. One method for rapidly determining these associations involves a sub-pooling strategy, amplification of the tags and hybridization to an array of oligonucleotides that are complementary to the tags (see Strathmann, M., U.S. Pat. No. 6,480,791 for a complete description). If the collection of cells is maintained as a single culture, another method is needed to isolate from the population the specific cell clone comprising the tagged-insertion element of interest.

A general method for selecting or enriching for a cell clone comprising a tagged insertion element (or any tagged component) exploits the mechanism of RNA Interference (Tijsterman M. et al., Annu. Rev. Genet. 36:489-519, 2002) to degrade a transcript which contains the tag sequence. Consider a tagged insertion element that comprises a selectable marker (for example, HSV thymidine kinase, gpt, etc., see Karreman, Nucleic Acids Res. 10:2508-2510, 1998) such that the transcript for the selectable marker contains the tag sequence. Such a marker is defined as a “tagged marker”. For illustrative purposes, it is simplest to think of the product of the tagged marker as a protein that confers a simple property on the cell, such as resistance to a chemical compound. One skilled in the art will recognize the product of the tagged marker may be for example, a subunit of a larger protein or may not be a protein at all, rather the product may be for example a nucleic acid that confers a selectable property on the cell. The tagged insertion element is easily constructed using standard recombinant techniques to place the tag, for example between a promoter and the coding sequence of the marker (5′-untranslated) or downstream of the coding sequence but before termination signals (3′-untranslated). This transcript will be degraded by introducing into the cell siRNA molecules that target the tag sequence. In other words, siRNA specific to the tag will downregulate the selectable marker in the cell. If loss of the marker confers a selectable phenotype on the cell, then only those cells that no longer produce the marker will survive. Starting with a population of cells, wherein each cell expresses a tagged marker, one can select or enrich for cells carrying a specific tagged marker by introducing into the cells siRNA directed to that one tag followed by the appropriate negative selection. Depending on the cell type, one may also introduce double-stranded RNA, short-hairpin RNA (shRNA), DNA vectors that result in sequence-specific (i.e. tag-specific) RNA inhibition, etc. Other examples of sequence-specific RNA inhibition include antisense oligonucleotides, microRNA (miRNA), ribozymes, etc. In fact, any tag-specific means to prevent or inhibit production of the tagged marker is suited to the selection scheme outlined above. Suitable markers include gpt, HSV-tk, etc (Karreman, Nucleic Acids Res. 10:2508-2510, 1998).

It is preferable to ensure the starting population of cells all express the marker gene therefore a preferred marker will also confer a positive advantage to the cells under different selective conditions (see for example, Karreman, Nucleic Acids Res. 10:2508-2510, 1998; Besnard, C. et al., Mol. Cell. Biol. 7:4139-4142, 1987; Wei, K. et al. J. Biol. Chem. 271:3812-3816, 1996). In this way, one can select for the marker (positive selection) before inducing RNA inhibition and selecting against the marker (negative selection). The result is a lower “background” of cells that survive the negative selection for reasons other than RNA inhibition. For example, a population of cells carrying a tagged gpt marker may be grown in the presence of HAT medium. After transfecting siRNA to one tag (or more), the growth media is changed to remove HAT and add 6-thioxanthine so only those cells that do not express gpt will survive (Besnard, C. et al., Mol. Cell. Biol. 7:4139-4142, 1987). Of course it may be adequate, for example, to introduce a second, different marker gene along with the tagged marker in the insertion element. In this way, a positive selection may be applied through the second marker while the negative selection is applied through the tagged marker. The second marker could be driven by a second promoter or it could be driven by the same promoter as the first marker to form a polycistronic transcript by, for example, introducing an internal ribosome entry site (IRES). In the case of a polycistronic transcript, both markers are subject to downregulation by the introduction of an siRNA to the tag sequence. Again, the goal is to reduce the “background” cells that survive the negative selection through means other than RNA inhibition.

It will be obvious to one skilled in the art that there are many variations of the RNAi selection for tagged markers described above. For example, the loss of the marker transcript need only produce a phenotype or characteristic that is distinguishable in some way from expression of the transcript. For example, the marker could be GFP (green fluorescent protein) and cells are sorted by FACS (fluorescence-activated cell sorting) to separate those cells that no longer fluoresce. The marker could be a transcription factor that inhibits expression of a cell surface antigen. Loss of the marker leads to expression of the surface antigen which allows isolation of cells by for example FACS, panning with antibodies to the surface antigen, etc.

More generally, one skilled in the art will recognize any means to modulate production of the tagged marker that depends on the sequence of the tag may be used to select from a population of cells comprising different tags those cells comprising a specific tag. For example, any tag-specific means to induce production of the tagged marker may be employed to select for the presence of the tagged marker. For example, triplex forming oligonucleotides, engineered zinc-finger binding proteins, etc. may be used as engineered transcription factors to modulate gene expression in a sequence (e.g. tag) specific manner (Visser, A. E. et al, Adv. Genet. 56:131-161, 2006; Gommans, W. M. et al, J. Mol. Biol. 354:507-519, 2005). In this context, the “tagged marker” comprises a marker and a tag that are not necessarily present on the same transcript. Rather, the tag is functionally linked to the transcript comprising the marker by the means to modulate production of the marker. It will be obvious to one skilled in the art how to tag a marker to make a tagged marker given the means to modulate the marker. Note, the term marker can be used to denote a gene or the product of that gene and the meaning is obvious to the skilled artisan from the context. By definition, a “tag-specific selection” refers to a means for modulating the activity of a tagged marker based on the sequence of the tag so that if the sequence of a second tag is substantially different, the tagged markers comprising the second tag will not be so modulated. The degree to which the sequence of two tags must differ depends on the means for modulating the activity of the tagged marker and is obvious to one skilled in the art. Examples of tag-specific selections include RNAi, miRNA, antisense oligonucleotides, ribozymes, etc.

The examples above describe an RNAi selection method wherein both the tag and the marker are introduced to the cell by some means such as, for example, transfection, transformation, infection, etc. A similar method may be applied to a population of cells in which only the marker is introduced exogenously. In the latter case, the tag is a genomic tag as described in U.S. Pat. No. 6,480,791. The genomic tag is determined by proximity to the insertion element in the genome. For example, standard gene-trap vectors can be used to produce a population of cells with random or quasi-random integration sites in genes throughout the genome. The vectors result in fusion transcripts between sequences present in the genome and a marker gene present in the vector. To select for cells in which a specific gene is “trapped”, one need only induce RNA interference to that specific gene followed by selection for loss of the marker. Depending on the cell type, RNA interference may be induced by introducing to the cells siRNA to the gene of interest, double-stranded cRNA to the gene, shRNA, etc. The specific gene will be downregulated by RNA interference (if it is expressed) but so too will be the gene fusion transcript. Loss of the fusion transcript results in loss of the marker which in turn allows the cell to survive the selection.

The RNAi selection methodology described above may also be used to select or enrich for homologous recombination between an exogenous construct and its homologous site in the genome. Typically, to perform gene targeting by homologous recombination a marker is ligated into genomic sequences in vitro by standard recombinant techniques. The resulting construct is introduced into cells followed by selection for the presence of the marker. In many cell types, the frequency of random integration of the construct in the genome is much greater than the frequency of homologous recombination. In a manner analogous to that described above for trapped genes, one can select or enrich for homologous recombination events by directing RNA interference to the gene designed to undergo targeting by homologous recombination. The targeting construct should be designed to produce a transcript that encodes the marker and carries additional sequence from the gene of interest. The additional sequence should not be part of the construct, rather it is incorporated when the construct undergoes homologous recombination. For example, in vertebrates the marker may be designed like the marker in a poly-A trap vector, which has a splice donor downstream of the marker. The targeting construct will have genomic sequence on either side of the marker to allow recombination with the endogenous gene. Transcription of the marker leads to splicing with downstream exons that by design are not part of the targeting construct. RNA interference may then be targeted to the downstream exon sequences (i.e. a downstream exon comprises the genomic tag). If the construct integrates randomly, then downstream sequences will not be present in the transcript which encodes the marker. Consequently, the marker will not be downregulated by RNA interference and selection against the marker will for example kill the cell. Only a construct which has integrated into the genome in an orientation that yields a fusion transcript between the marker and the downstream sequences will be subject to RNA interference. Only this orientation of the integrated construct will permit the cell for example to survive under the negative selection conditions. This orientation is most likely to occur as the result of homologous recombination. Therefore, by choosing the appropriate marker gene (e.g. gpt, HSV-tk, etc.) and using the RNAi selection scheme one can select or enrich for homologous recombination events. In some cell types, it may be necessary to include only intron sequences in the targeting construct or design the fusion transcript to include upstream exons to target for RNAi so that the effects of transitive RNAi on randomly integrated constructs may be avoided (Sijen, T. et al., Cell, 107:465-476, 2001).

The RNAi selection scheme as practiced with a tagged marker is a general method for selecting or enriching for a specific tagged cell from a population of tagged cells. This scheme can have utility in addition to the gene targeting applications described above. For instance, a certain property or phenotype may vary among tagged cells. This variation may be monitored in the population under some set of experimental conditions which could lead to the identification of only a small subset of tagged cells of interest. This subset may be quickly isolated from the population by applying the RNAi selection scheme. For example, a large collection of tagged cells are created as described above by using tagged insertion elements. The insertion elements are designed to integrate at only one location in the genome by using for example site-specific recombination. The population of cells is subjected to chemical mutagenesis so that a small number of mutations are introduced at random in each tagged cell. The population is exposed to a drug for some period of time and hypersensitivity to the drug is investigated. By using microarrays comprising oligonucleotides complementary to the tags, it is possible to monitor the loss from the population of certain tagged cells as described by Mazurkiewicz et al. (Nat. Rev. Genet. 7:929-39, 2006). The cells which show hypersensitivity may be isolated from the untreated population of cells using the RNAi selection scheme (i.e. siRNA directed to the appropriate tag) and investigated further.

### 7. EXAMPLES

### Example 1

### Gene Trapping—Identification and Isolation of a Specific Clone

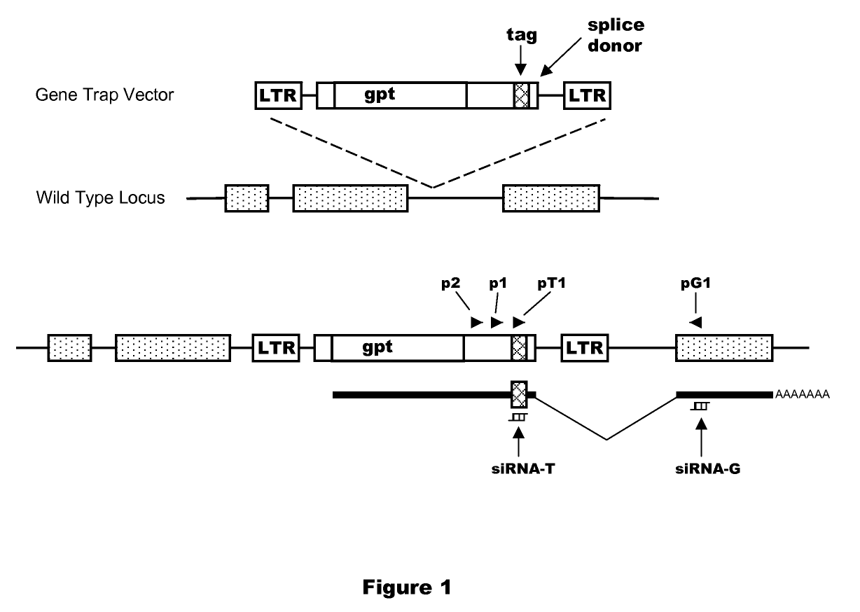

A collection of gene trap vectors is made by standard recombinant techniques as shown in FIG. 1. The vector backbone is a 3′ gene trap (i.e. polyA trap) vector described in U.S. Pat. No. 6,080,576. The selectable marker is gpt (xanthine-guanine phosphoribosyl transferase). The presence of gpt can be both selected for and against (see U.S. Pat. No. 6,689,610 for selective agents and preferred concentrations). The vectors are identical except for a 25 basepair sequence indicated as the “tag” in the figure. The tags are first synthesized as effectively random sequences and then ligated into a restriction site in the parent vector, which lacks a tag, to generate the collection of vectors.

This collection of vectors is then packaged into retroviral particles by standard means as described in U.S. Pat. No. 6,080,576 (see also Viral Vectors for Gene Therapy: Methods and Protocols Ed. Machida, C. A., Humana Press, New Jersey (2003); Gene Delivery to Mammalian Cells: Volume 2: Viral Gene Transfer Techniques Ed. Heiser, W. C., Humana Press, New Jersey (2004); The Centre for Modeling Human Disease Gene Trap resource, http://www.cmhd.ca/genetrap/protocols.html). Supernatant from the packaging cells is added to embryonic stem cells for 16 hours and the cells are grown in the presence of gpt selection reagent (Millipore, Billerica, Mass.) according to the manufacturer's instructions (see also U.S. Pat. No. 5,627,033) for 10 days. Surviving cells (i.e. those cells expressing gpt) are isolated into 100 pools of about 1000 distinct clones per pool. Each pool is grown up and subjected to automated RNA isolation and reverse transcription by standard protocols (see U.S. Pat. No. 6,080,576) to make cDNA. cDNA from each pool is combined to make a single pool of cDNA products from about 100,000 distinct clones. The tags are PCR amplified from the single pool of cDNA products using a 3′-RACE protocol. Two rounds of PCR with nested primers (see p1 and p2 in FIG. 1) are performed as described (ibid).

The amplified 3′-RACE PCR products containing the tags are sequenced using the Genome Sequencer FLX System instrument sold by Roche (Indianapolis, Ind.) using protocols supplied by the manufacturer. The sequence information indicates where the gene trap vector has inserted in the genome and the unique tag associated with each insertion site.

A specific cell clone is isolated using the unique tag (Tag1) associated with the clone. First, a PCR primer is designed to hybridize to Tag1 in the orientation shown in FIG. 1 (see pT1). PCR is performed with pT1 and the gene specific primer, pG1 (see FIG. 1), on the cDNA isolated from each of the 100 pools of clones described above. The presence of an amplification product identifies the pool to which the specific cell clone belongs.

The specific clone is isolated from the identified pool of about 1000 clones by using the RNAi selection method. An siRNA targeted to the tag sequence (siRNA-T, in FIG. 1) is synthesized (Qiagen, Valencia, Calif.) and introduced by transfection into the appropriate pool of 1000 clones using the HiPerFect Transfection Reagent (Qiagen, Valencia, Calif.) according to the manufacturer's instructions. After 2 days the cells are again transfected with siRNA as above and transferred to fresh media supplemented with 100 μM 6-thioxanthine to select for the loss of gpt. After three days, the surviving cells are transferred to fresh media and grown in the absence of selective pressure for three days. Finally the cells are transferred to media supplemented with gpt selection reagent to eliminate any cells that survived 6-thioxanthine treatment by losing the gpt gene (by for example chromosome loss or mutation of the gene). The resulting cells are highly enriched for the cell clone carrying the specific tag, Tag1.

Alternatively, the RNAi selection procedure as described above is performed with siRNA targeted to the gene in which the gene trap vector resides. In this case, a genomic tag is utilized for the procedure and siRNA-G (see FIG. 1) is introduced into the pooled cells by transfection.

### Example 2

### Homologous Recombination—Selection for the Correct Recombinant Events

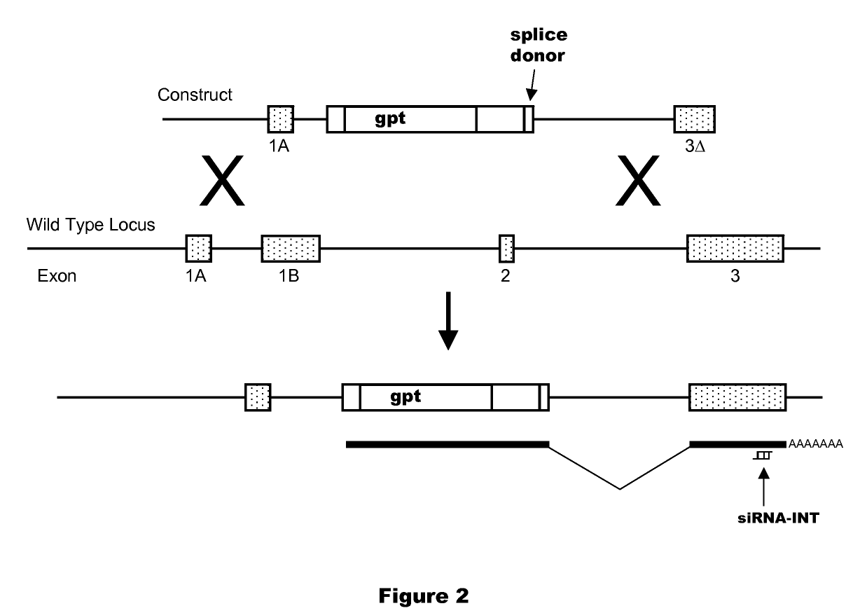

Capecchi and Thomas describe the disruption of the INT-2 gene in mouse ES cells by homologous recombination with an introduced construct (U.S. Pat. No. 5,464,764). The construct shown in FIG. 2 is made using standard recombinant techniques. The construct contains the gpt gene from Example 1 above flanked on both sides by sequences derived from the INT-2 gene so that the last exon (3Δ in FIG. 2) is truncated (see Example 1 and FIGS. 5A, 5B & 5C in U.S. Pat No. 5,464,764; and Mansour, S. L. & Martin, G. R., EMBO, 7:2035-2041, 1988). The purified construct is introduced into mouse ES cells by transfection as described (see U.S. Pat No. 5,464,764). The transfected cells are grown in the presence gpt selection reagent as described above to select for the expression of gpt. Most of the surviving cells are due to random integration of the construct into the genome. Only a small percentage of the cells have incorporated gpt by homologous recombination with the endogenous INT-2 gene. These rare recombination events are selected using the RNAi selection scheme described above. The siRNA shown in FIG. 2, siRNA-INT, is designed to target a portion of the INT-2 transcript that is not present in the construct shown in FIG. 2. This siRNA is introduced by transfection and cells are selected for the loss of gpt function as described above in Example 1. After this negative selection is performed, the surviving cells are again subjected to a positive selection for gpt function also described in Example 1. The cells that survive this procedure are highly enriched for the integration of the construct into the INT-2 gene by homologous recombination.

### 8. INCORPORATION BY REFERENCE

The contents of all cited references (including literature references, patents, and patent applications) that may be cited throughout this application are hereby expressly incorporated by reference.

### 9. EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

I claim:

1. A method for enrichment of cells comprising a tagged marker with a first tag from a collection of cells comprising the tagged marker with a second or no tag by modulating the activity of the tagged marker with a tag-specific selection.

2. The method of claim 1, wherein the collection of cells comprising the tagged marker is produced by introducing a construct comprising a marker and sequences homologous to the cell's genome such that the construct recombines with the genome to produce the tagged marker with the first tag.

3. The method of claim 2, wherein the first tag is a genomic tag and the construct does not comprise the first tag.

4. The method of claim 1, wherein the collection of cells comprising the tagged marker is produced by inserting into the cell's genome a plurality of constructs comprising a marker and a plurality of tags.

5. The method of claim 4, wherein the constructs are tagged insertion elements.

6. The method of claim 1, wherein the tag-specific selection comprises RNAi.

7. The method of claim 6, wherein RNAi is targeted to the first tag.

8. The method of claim 7, wherein RNAi is induced by introducing synthetic siRNA or shRNA into the cells.

9. The method of claim 1, wherein the tag-specific selection comprises miRNA.

10. The method of claim 1, wherein the tag-specific selection comprises antisense compounds.

11. The method of claim 1, wherein the tag-specific selection comprises ribozymes.

12. The method of claim 1, wherein the tag-specific selection comprises triplex-forming oligonucleotides.

13. The method of claim 1, wherein the tag-specific selection comprises engineered transcription factors.
